(19) United States
(12) Patent Application Publication
(10) Pub. No.: US 2013/0018417 A1
(43) Pub. Date: Jan. 17, 2013

(54) SPINAL ALIGNMENT DEVICE AND METHOD

(71) Applicants: David Goldberg, Piedmont, SC (US); Jason Belton, Norton, OH (US); Craig Rae, Salisbury, NC (US)

(72) Inventors: David Goldberg, Piedmont, SC (US); Jason Belton, Norton, OH (US); Craig Rae, Salisbury, NC (US)

(73) Assignee: SMC LTD., Somerset, WI (US)

(21) Appl. No.: 13/180,771

(22) Filed: Jul. 12, 2011

(51) Int. Cl. A61F 5/00

(52) U.S. Cl. 606/240

(57) ABSTRACT

An adjustable head and neck support device with an adjustable head rest assembly and an adjustable neck rest assembly that attach to and move with respect to a base. The adjustable neck rest assembly is configured to move and non-fixedly lock on a z-axis and to rotate about non-fixedly lock on a y-axis. The head rest support assembly rotates about non-fixedly lock on an x-axis. The adjustable head rest assembly is configured to move and non-fixedly lock on the z-axis and to rotate about the x-axis.

### FIELD OF THE INVENTION

The invention relates generally to a device and method of utilizing an adjustable device to support the head and neck of a user capable of providing alleviation to tension and stress on the neck muscles, tendons, spine and joints caused by tight muscles and poor spinal alignment. More specifically it relates to an adjustable neck and head support device for progressively aligning an individual's spine.

### BACKGROUND OF THE INVENTION

The vertebrae of an individual's spine should be in “neutral alignment” in order to increase an individual's comfort, and reduce neck and back strain. Neck and head support adjustment devices are well known in the art. However, they are not easily portable, expensive and cannot be easily adjusted.

Accordingly, it is desirable to have an adjustable neck and head support device, which can be quickly and easily adjusted by the individual or care provider that is portable, inexpensive and adjustable.

### SUMMARY OF THE INVENTION

Accordingly, the present invention overcomes the limitations of the prior art by providing a unique and useful device and method of utilizing an adjustable neck and head support device to support the head and neck capable of providing alleviation to tension and stress in the neck muscles, tendons, spine and joints caused by tight muscles, poor spinal alignment, and the like. The adjustable neck and head support device is easy and safe to use, easily portable and stored, inexpensive and easy to adjust.

Consequently, the following presents a simplified summary of the invention in order to provide a basic understanding of some aspects of the invention. This summary is not an extensive overview of the invention. It is intended to neither identify key or critical elements of the invention nor delineate the scope of the invention. Its purpose is to present some concepts of the invention in a simplified form as a prelude to the more detailed description that is presented later.

It is an object of the present invention to provide an adjustable neck and head support device, which can be quickly and easily adjusted by an individual, a family member, a health care provider and the like according to an embodiment of the invention.

It is a further object of this invention to provide an adjustable neck and head support device, which is easily portable according to another embodiment of the invention.

It is another object of the present invention to provide an adjustable neck and head support device, which is lightweight, and the like.

It is yet another object of this invention to provide an adjustable neck and head support device, which is easy to clean, yet according to another embodiment of the invention.

It is a further object of this invention to provide a lightweight and portable adjustable neck and head support device, according to another embodiment of the invention.

### BRIEF DESCRIPTION OF THE DRAWINGS

Embodiments of the invention will now be described with reference to the accompanying drawings.

FIG. 1 illustrates a top and front/side perspective view of an adjustable neck and head support device with a cushioned neck assembly at its highest vertical position according to an embodiment of the invention.

FIG. 2 illustrates a top view of a portion of an adjustable neck and head support device with a slotted base showing four slots according to another embodiment of the invention.

FIG. 3 illustrates top and side perspective view of an adjustable neck and head support device with four visible arm slots yet according to another embodiment of the invention.

FIG. 4 illustrates a top and side perspective view of an adjustable neck and head support device with a cushioned neck assembly at an adjusted vertical position according to another embodiment of the invention.

FIG. 5 illustrates a top and back perspective view of an adjustable neck and head support device with a cushioned neck assembly with a head cushion being installed on a head pad being installed on according to an embodiment of the invention.

FIG. 6 illustrates a side perspective view of FIG. 5 illustrates a top and side perspective view of an adjustable neck and head support device with a cushioned head assembly at its highest vertical position according to an embodiment of the invention.

FIG. 7 illustrates a side perspective view of an adjustable neck and head support device with a cushioned neck assembly at its lowest vertical position according to an embodiment of the invention.

FIG. 8 illustrates a side perspective view of an adjustable neck and head support device with a cushioned neck assembly, according to yet another embodiment of the invention.

FIG. 9 illustrates a functional block diagram of adjusting an adjustable neck and head support device with a cushioned neck assembly into one position, according to yet another embodiment of the invention.

### DETAILED DESCRIPTION OF THE INVENTION

In a head and neck support device, if the neck and/or head support in the therapeutic device is too high or too low, the vertebrae in the neck will be out of alignment with the vertebrae in the rest of the spine. This may aggravate neck and back problems. The level of neck and/or head support device required will vary according to body size, spinal alignment, and the like.

The invention relates generally to an adjustable head and neck support device capable of providing alleviation of tension and stress on the neck muscles, tendons, and joints caused by tight muscles, poor spinal alignment and the like. This is a system and method to utilize the device to help improve poor posture that can result from many of today's lifestyles. For example, sitting, poor posture and staring into a computer screen, watching television and other passive activities that can result in a spinal misalignment and pain.

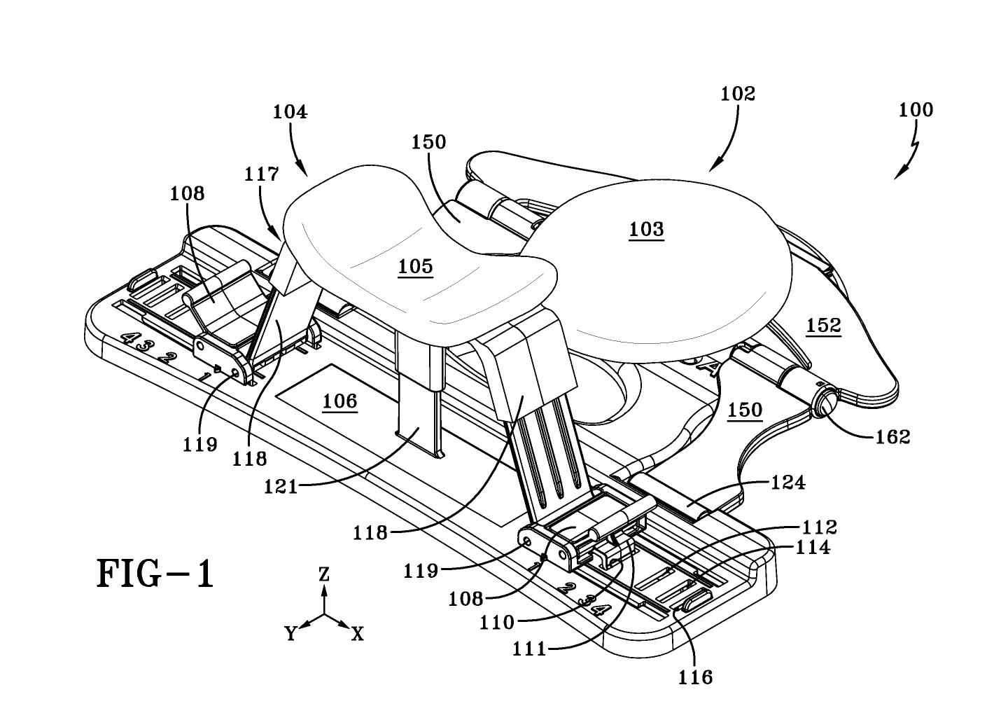

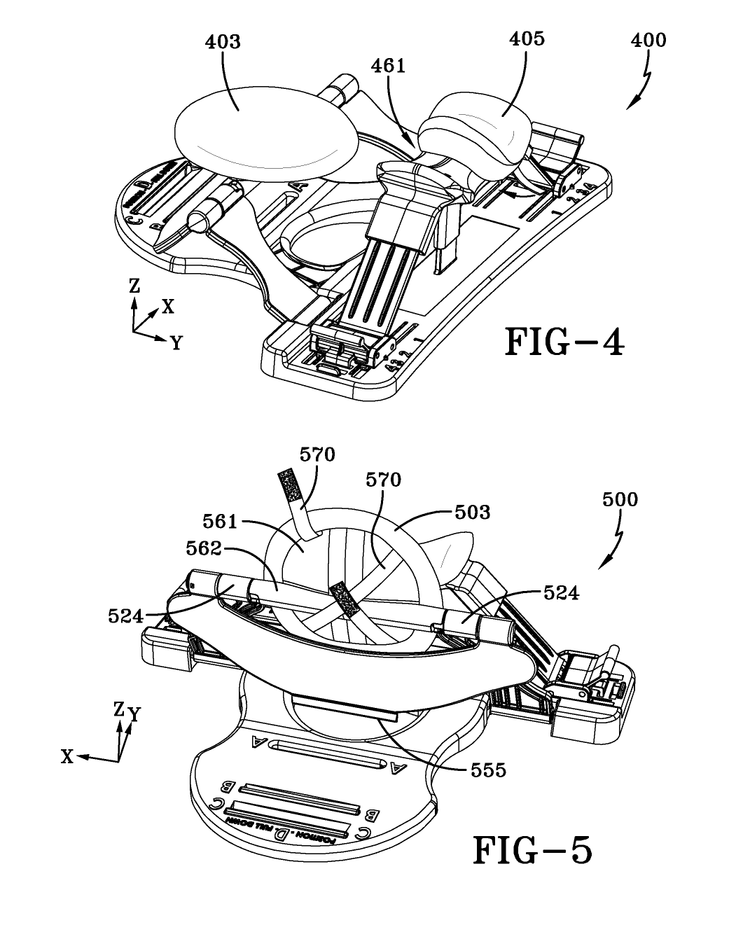

Referring now to the drawings an adjustable head and neck support device 100 (FIG. 1) is configured with an adjustable head rest assembly 102 and an adjustable neck rest assembly 104 that are configured to attach to and move with respect to a base 106. The base 106 is attached to headrest arms 150 that rotate within base hinges 124. A user's neck comes into contact with a cushioned neck pad cover 105 that that can be located in the middle or another location of the user's neck. A cushioned neck pad 405 (FIG. 4) is stretched over a neck pad 460 in the cushioned neck rest assembly 104. The adjustable head pad assembly 102 rotates about a head pad axle 162 that rotates within the base hinges 124 and a head pad cover 503 (FIG. 5) is held to a head pad axle 562 by hook and loop straps 570. Other techniques and devices comprising buttons and loops, adhesive tape, snaps, eyelets, and the like can be utilized to hold a pad in place. Numerous options are available for moving the adjustable head rest assembly 102 and the adjustable neck rest assembly 104 to accommodate the individual user's comfort, by those of skill in the art.

The devices illustrated in FIGS. 1-8 can be made out of various materials comprising, plastics, polymers, PVC, ABS, wood, metal, metal, composites, combinations thereof and other materials known by those of skill in the art.

The inventors considered that other embodiments of the present invention may be utilized with a cushioned neck assembly and a cushioned head assembly without departing from the spirit and the scope of the invention. The cushioned neck assembly and the cushioned head assembly can be utilized with equipment comprising electronic heaters, cooling components, vibration devices, load cells, speakers, medicine pads, ointments, aromatherapy pads, pads filled with materials such as gels or beads, pads that are inflatable to increase or decrease pressure, pad foams comprising varying density and including advanced foams, rubber, etc. The electronic devices can use power sources comprising AC power, DC power, battery power, and the like known by those of skill in the art.

Although the cushioned pads 103 and 105 depicted show a neck pad cover 480 (FIG. 4) and a head pad cover 580 (FIG. 5) with cloth fabric, the covers 403 and 405 can be made using any one of materials used by one of ordinary skill in the art, comprising, vinyl, gauze, disposable tear off paper, etc. In addition, the covers 403 and 405 can comprise washable, dry cleanable; throw away devices, etc., to use in between different patients/users, etc.

In this embodiment, two lock and release levers 108 can be part of the adjustable neck rest assembly 104. The levers 108 can be connected to two neck adjustment arms 118. An adjustable vertical component 121 ensures that the neck rest assembly 104 travels on the z-axis only. However, one skilled in the art can devise a neck rest assembly that can be locked in any 3D position with the headrest assembly horizontal or tipped at an angle, for example.

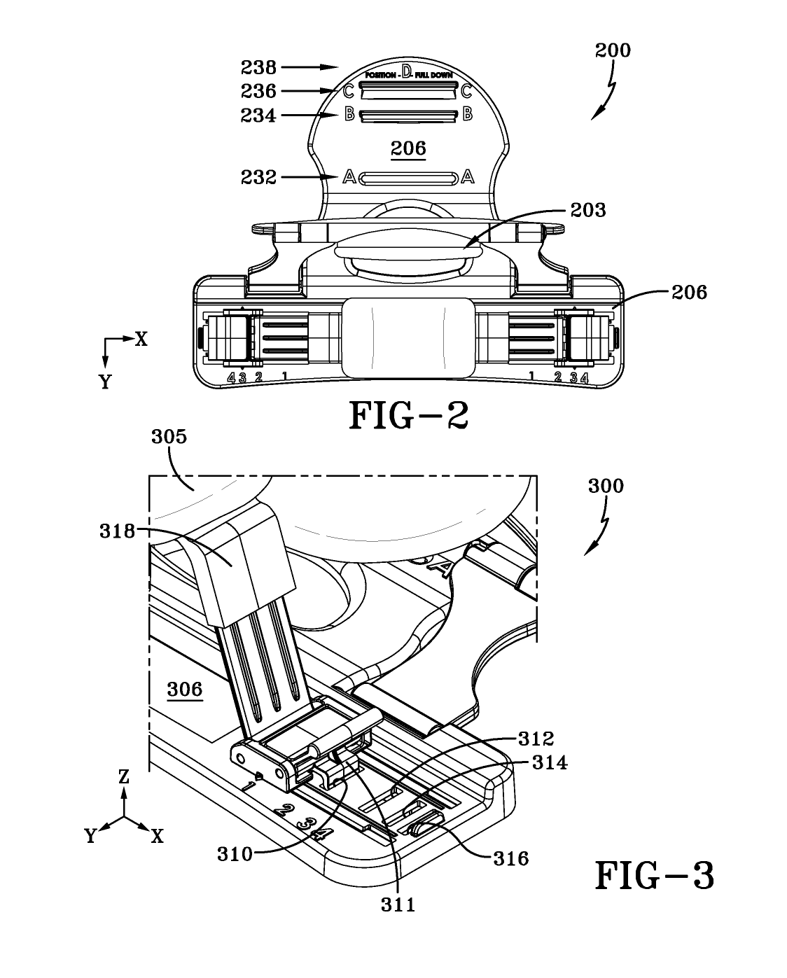

The adjustable head rest assembly 102 can be raised to its highest position which is done by inserting headrest tilt plate tab 555 (FIG. 5) into a first base slot 232 (FIG. 2) with the headrest arms 150 rotated into position. The tab 555 is not as wide as a given slot width and the tab width is less than the slot width. The tab length is less than the slot depth so that when the tab 555 is inserted into the slot it locks the assembly 102 in place. Once the tab 555 is placed in a given slot the user can apply pressure in a downward (−y direction) fashion to either the adjustable head rest assembly 102 and/or the adjustable neck rest assembly 104 to ensure that they are securely set and that they don't slide or move when the pressure is applied. The device comprises numerous neck arm base slots 110, 112, 114 and 116 that are utilized to adjust the height of the adjustable neck rest assembly 104.

The arms 118, at hinges 119 near the base 106 are free to rotate both at the hinges 119 and at the lock and release lever hinges 119.

The device 100 can be placed on the floor, on a treatment table, on a comfortable supporting surface such as a chair or a couch so that the user can slowly lower themselves onto the device so that their head makes contact with the adjustable head rest assembly 102 and their neck with the adjustable neck rest assembly 104. In another embodiment, the device 100 can be permanently or non-fixedly attached to a treatment device such as a treatment bed, chair or other treatment devices well known by those of skill in the art. The user at the time of using the device 100 can use other therapeutic devices such as a pillow, wedge, etc., under their knees to help relax the lower back.

In another embodiment of the present invention, the device 100, the adjustable head rest assembly 102 and the adjustable neck rest assembly 104 can be adjusted so that the user finds a level which is comfortable but also provides a slight stretch on the neck and the spinal cord. The device 100 is designed so that the user can move up in levels of advancement as they become more conditioned to the device 100. In addition, the inventors envisioned different adjustable size and shape head and neck pads and assemblies where the size and shape can be adjusted without departing from the spirit and the scope of the invention, known by those of skill in the art.

Although the device 100 is shown with a fixed distance, in the y direction between the cushioned head pad cover 103 and the cushioned neck pad cover 105 in the various positions shown in Table 1 below, those skilled in the art will recognize many modifications that may be made to this configuration, without departing from the scope or spirit of what is described herein. For example, a device can be made by those skilled in the art so that the base is segmented so that it expands or shortens in length in the y direction. Also, the device can be manufactured so that both the adjustable head rest assembly and adjustable neck rest assembly can be rotated independently around the z-axis to accommodate users with various spinal conditions. These various configurations and designs are well known by those of skill in the art. In addition, a device can be made without slots but rather lock and release levers that can be locked in any position along the rail, using a device comprising an overturning locking cam, a threaded rod, a rack and pinion, for example.

The inventors also contemplated that the concepts of the present invention may be utilized with other types and configurations of moving the various assemblies without departing from the spirit and the scope of the invention. For example, the device can utilizes equipment comprising motors, linear actuators, springs, torsion devices, threaded shafts, load cells, proximity sensors, or sensor electronics, to adjust the head and neck assembly, as well as other components. Also, the device assembly locations could be digitally set using an electronic programmable device with software that drives the head and neck assemblies with the patients head and neck on the device controlled by a computer program based on variables comprising patients spine curvature, load cells, proximity sensors, x-ray measurements, a doctor's prescription, biofeedback, proximity sensors, load or force sensor readings, and the like. These various configurations are well known by those of skill in the art.

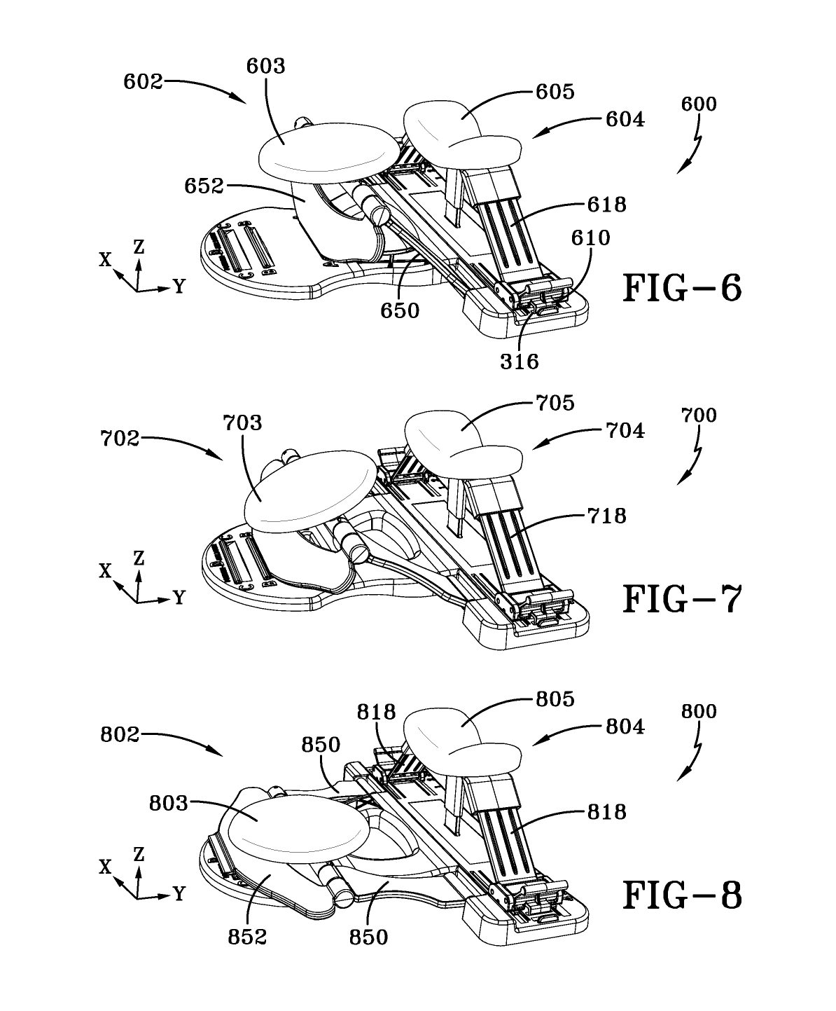

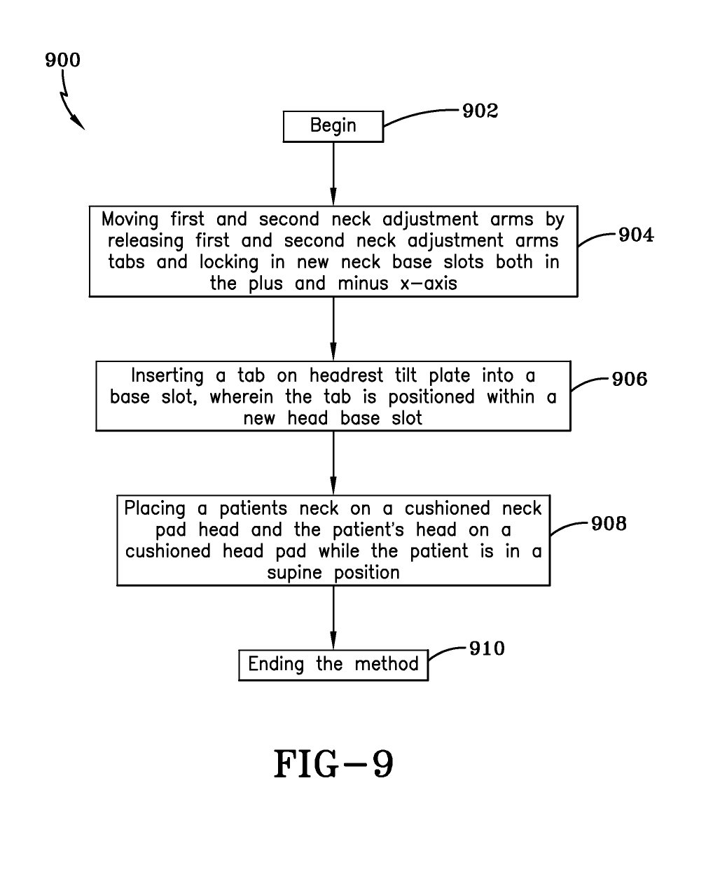

In another embodiment of the present invention, FIG. 2 illustrates a device 200 with various head base slots 206. The first base slot 232 is utilized by placing a tab 555 (FIG. 5) on a headrest tilt plate 652 (FIG. 6) and headrest arms 450 (FIG. 450) and rotating into position when a user wants a cushioned head assembly locked in a z-position. A second base slot 234 and third base slot 236 are also utilized for holding a cushioned head assembly 203 at a given height in the z-direction. A fourth base position 238 does not involve a slot but rather is the position 238 when the cushioned head assembly 203 is at its lowest position (full down) and the headrest tilt plate 652 is resting on a base 206.

In yet another embodiment of the present invention, FIG. 3 illustrates a device 300 with various slots in a base 306 used to lock arms 318. A first arm base slot 310 is utilized by placing a tab 311 in the slot 310 when a user wants a cushioned neck assembly 305 in the highest z-direction position. A second arm base slot 312 and third arm base slot 314 are also utilized for holding the cushioned neck assembly 305 at a given height in the z-direction. Fourth arm base slot 316 is utilized when the user wants the cushioned neck assembly 305 at its lowest position.

Another embodiment of the present invention is illustrated in FIG. 4. A device 400 comprises a neck pad 405 with the user attaching the neck pad cover 405 wherein the neck pad cover 405 and a head cover 403 can comprise materials such as cloth fabric, any one of materials used by one of ordinary skill in the art, comprising, vinyl, gauze, paper, etc. In addition, the neck pad cover 405 can be washable, dry cleaned, a throw away device to use in between different patients/users, etc. Although this embodiment illustrates the neck pad 460 as non-rotating, the inventors contemplate neck pads that are rotatable utilizing techniques well know by those of skill in the art.

Yet another embodiment of the present invention is illustrated in FIG. 5. A device 500 comprises a head pad 561 with the user attaching a head pad cover 503 wherein the head pad cover 503 and a neck pad cover 505 can comprise materials such as cloth fabric, any one of materials used by one of ordinary skill in the art, comprising, vinyl, gauze, paper, etc. In addition, the covers 503 and 505 can be washable, dry cleaned, a throw away device to use in between different patients/users, etc. In this embodiment, the head pad 561 is attaching the head pad cover 503 to the head pad 561 utilizing the hook and loop material 570 wrapped around the axle 562 where the head pad 561 rotates about base hinges 524. This arrangement allows a head pad assembly 502 to rotate about the axle 562 with headrest arms 850 rotated and locked into the proper location.

A device 600, 700 and/or 800, for example can be set in numerous configurations for the patient/user. Those configurations are outlined in Table 1 below. Only a few of those configurations illustrated in the drawings and Table 1 are described for clarity below. Although the various configurations are shown in Table 1, many more configurations are contemplated by the inventors and known by one skilled in the art. Table 1 is only a small sample of possible configurations.

In yet another embodiment of the present invention, a cushioned neck rest assembly 604 of the device 600 (FIG. 6, Table 1, “Starting Position”, Set-up 1) is moved to lowest neck rest position. This is done by moving arms 618 in a released state and locking the lock and release levers 108 into the slots 316 (FIG. 3) where the lock and release levers 108 is again pushed down and a head tab (not shown is located on a head rest assembly (602). In the starting position, the cushioned head rest assembly 602 is at its highest possible height for the device 600 and the cushioned neck rest assembly 604 is at its lowest possible height for the device 600. As illustrated in FIG. 6, the device 600 is configured so a head cover 603 and a neck cover 605 are at approximately the same height. In this configuration, a user's lower neck and a base of a user's head are at approximately the same height in the positive z-direction.

Another embodiment of the present invention is shown in FIG. 7, wherein the device 700 can be positioned in an “intermediate position” 701. Referring to Table 1, set-up 6 the “intermediate position” 701 is configured by moving both neck adjustment arms 718 locked and/or positioned within the third arm slot 314 (FIG. 3), and a tab 555 (FIG. 5) on a base component 753 locked and/or positioned within a second base slot 234 (FIG. 2). In the intermediate position (Table 1, Set-Up 6), a cushioned head cover assembly 702 (with cover 703) is lower (and shown rotated), and a cushioned neck assembly 704 (with cover 705) is higher, than depicted in FIG. 6. In this configuration, a user's lower neck and a base of a user's head are at different heights in the positive z-direction, stretching the user's neck and adjusting the user's spine.

An example of another embodiment of the present invention is shown in FIG. 8, wherein the adjustable device 800 can be positioned in an “advanced position” 801. Referring to Table 1, set-up 9 the “advanced position” 801 is configured by moving both neck adjustment arms 818 with corresponding tabs (not shown) locked and/or positioned within the second arm base slot 312 (FIG. 3), and a headrest tilt plate 752 with corresponding tab 855 is positioned in the fourth base position 238 (FIG. 2). In the advanced position 801, a cushioned head cover 803 is flat at its lowest position, and a cushioned neck assembly 804 and assembly 802 is lower, than depicted in FIG. 6. As illustrated in FIG. 8, the adjustable device 800 is configured so the cushioned head cover 803 is lower than the cushioned neck cover 805. In this configuration, a user's neck and base of user's head are at different heights in the positive z-direction, stretching the user's neck and re-aligning the user's spine to a greater extent than FIGS. 6 and 7.

A method 900 of positioning the adjustable device 800 in an intermediate position will be discussed with respect to changing the position of the adjustable head and neck support device 600 in FIG. 6 to the position of the device 700 in FIG. 7. As illustrated in FIG. 6, and discussed supra, the neck rest assembly 604 of the device 600 (FIG. 6, Table 1, “Starting Position”, Set-up 1) is in the lowest neck rest position. In the starting position, the cushioned head assembly 602 is at its highest possible height for the adjustable device 600 and the cushioned neck rest assembly 604 is at its lowest possible height for the device 600.

To go from the position in FIG. 6 to FIG. 7 the first and second neck adjustment arms are moved by releasing a first and second neck adjustment arms tabs 111 from the two slots 316 by releasing the two lock and release levers 108. The tabs 111 are then moved to the two slots 314 and locked in place by releasing the two lock and release levers 108. The head rest assembly tab 555 on a headrest tilt plate 152 is then taken out of the head base slot 234 and placed in the base slot 236.

The neck adjustment arms 818 with the corresponding tabs 111 are moved from the slots 314 and locked and/or positioned in the slot 312 (FIG. 3). The headrest tilt plate tab 555 is then moved from the slot 236 into the fourth base position 238. The patient's neck and head is placed on the cushioned neck pad cover 105 and the cushioned head pad cover 103, respectively, while the patient is in a supine position, ending the method.

Although the invention has been illustrated and described with respect to one or more embodiments, implementations, alterations, and/or modifications may be made to the illustrated examples without departing from the spirit and scope of the appended claims. In particular regard to the various functions performed by the above described components or structures (assemblies, devices, systems, etc.), the terms (including a reference to a “means”) used to describe such components are intended to correspond, unless otherwise indicated, to any component or structure which performs the specified function of the described component (e.g., that is functionally equivalent), even though not structurally equivalent to the disclosed structure which performs the function in the herein illustrated exemplary implementations of the invention. In addition, while a particular feature of the invention may have been disclosed with respect to only one of several implementations, such feature may be combined with one or more other features of the other implementations as may be desired and advantageous for any given or particular application. Furthermore, to the extent that the terms “including,” “includes,” “having,” “has,” “with,” or variants thereof are used in either 5 the detailed description and the claims, such terms are intended to be inclusive in a manner similar to the term “comprising”.

TABLE 1

| | | Neck Rest | Head Rest | | Neck Rest | Head Rest |
|---|---|---|---|---|---|---|
| | | Assembly | Assembly | Alternate | Assembly | Assembly |
| Set-Up | | Slot | Slot | Set-up | Slot | Slot |
| | Stage 1 | | | | | |
| | (Beginner) | | | | | |
| 1 | “Starting | Forth Arm | First Base Slot | N/A | N/A | N/A |
| | Position” | Slot 316 | 232 | | | |
| | (FIG. 6) | (FIG. 6) | (FIG. 6) | | | |
| 2 | Step 2 | Forth Arm | Second Base | OR | Third Arm | First Base |
| | | Slot 316 | Slot 234 | | Slot 314 | Slot 232 |
| 3 | Step 3 | Forth Arm | Third Base | OR | Second Arm | First Base |
| | | Slot 316 | Slot 236 | | Slot 312 | Slot 232 |
| 4 | Step 4 | Forth Arm | Forth Base | OR | First Arm | First Base |
| | | Slot 316 | Position 238 | | Slot 310 | Slot 232 |
| | Stage 2 | | | | | |
| | (Intermediate) | | | | | |
| 5 | Step 1 | Third Arm | Second Base | OR | Second Arm | Second |
| | | Slot 314 | Slot 234 | | Slot 312 | Base Slot |
| | | | | | | 234 |
| 6 | Step 2 | Third Arm | Third Base | OR | First Arm | Second |
| | (FIG. 7) | Slot 314 | Slot 236 | | Slot 310 | Base Slot |
| | | (FIG. 7) | (FIG. 7) | | | 234 |
| 7 | Step 3 | Third Arm | Forth Base | N/A | N/A | N/A |
| | | Slot 314 | Position 238 | | | |
| | Stage 3 | | | | | |
| | (Advanced | | | | | |
| | Position) | | | | | |
| 8 | Step 1 | Second Arm | Third Base | N/A | N/A | N/A |
| | | Slot 312 | Slot 236 | | | |
| 9 | Step 2 | Second Arm | Forth Base | N/A | N/A | N/A |
| | (FIG. 8) | Slot 312 | Position 238 | | | |
| | | (FIG. 8) | (FIG. 8) | | | |
| | Stage 4 | | | | | |
| | (Expert | | | | | |
| | Position) | | | | | |
| 10 | Step 1 | Second Arm | Third Base | N/A | N/A | N/A |
| | | Slot 312 | Slot 236 | | | |
| 11 | Step 2 | Second Arm | Forth Base | N/A | N/A | N/A |
| | | Slot 312 | Position 238 | | | |

What is claimed is:

1. An adjustable head and neck support device, comprising;
an adjustable head rest support assembly and an adjustable neck rest assembly configured to attach to and move with respect to a base; and
wherein the adjustable neck rest assembly is configured to move up and down on a z-axis and non-fixedly lock on the z-axis;
wherein the head rest support assembly is configured to move up and down on a z-axis, to move along the y-axis and is further configured to rotate around the x-axis;
wherein the head rest support assembly is further configured to lock non-fixedly lock in the y-z plane.

2. The adjustable head and neck support device of claim 1,
wherein the base is attached to head rest arms that rotate around respective base hinges; and
wherein the rotational head pad rotates about a head pad axle that rotates within the base hinges.

3. The adjustable head and neck support device of claim 1, wherein the base, the head rest arm, the head pad, the neck pad, lock and release levers; and the neck adjustment arms are made of materials comprising plastics, PVC, ABS, wood, metal, metal, composites and combinations thereof.

4. The adjustable head and neck support device of claim 1, wherein a head pad cover is stretched over the head pad in the head rest support assembly; and
wherein neck pad cover is stretched over a neck pad in a cushioned neck pad.

5. The adjustable head and neck support device of claim 1, wherein the head pad axle is held by devices comprising hook and loop straps, buttons and loops, adhesive tape, snaps and eyelets.

6. The adjustable head and neck support device of claim 1, wherein the cushioned neck assembly and the cushioned head assembly can be utilized with equipment comprising electronic heaters, cooling components, vibration devices, load cells, speakers, medicine pads, ointments, aromatherapy pads, pads filled with materials such as gels or beads, pads that are inflatable to increase or decrease pressure, pad foams comprising varying density and including advanced foams and rubber; and
wherein the electronic devices can use power sources comprising AC power, DC power and battery power.

7. The adjustable head and neck support device of claim 1, wherein the neck pad cover and the head pad cover, wherein the covers are made using materials comprising, cloth fabric, vinyl, gauze and paper;
wherein the covers and can comprise washable, dry cleanable and disposable devices.

8. The adjustable head and neck support device of claim 1, wherein the device can be placed or mounted fixedly or non-fixedly on a surface comprising the floor, a treatment table, a supporting surface such as a chair and a couch so that the user, with or without assistance can lower themselves onto the device so that their head makes contact with the adjustable head rest assembly 102 and their neck with the adjustable neck rest assembly 104.

9. An adjustable head and neck support device, comprising:
an adjustable head rest assembly and an adjustable neck rest assembly that are configured to attach to and move with respect to a base; and
wherein the adjustable neck rest support assembly is configured to move and non-fixedly lock at any position in its range of motion in axes comprising x, y and z;
wherein the head rest support assembly is configured to move and non-fixedly lock at any position in its range of motion in axes comprising x, y and z.

10. The adjustable head and neck support device of claim 9, wherein the device utilizes devices comprising motors, linear actuators, springs, torsion devices, threaded shafts, load cells, proximity sensors, sensor electronics or a combination thereof to adjust the head and neck assembly;
wherein the device locations are set using apparatus comprising an electronic programmable device with software, apparatus controlled by a computer program based on variables comprising patients spine curvature, x-ray measurements, a doctor's prescription, biofeedback, proximity sensors and load, force sensor readings or a combination thereof.

11. The adjustable head and neck support device of claim 9, wherein the cushioned neck pad and the cushioned head pad can be utilized with equipment or electronic devices comprising electronic heaters, cooling components, vibration devices, load cells, speakers, medicine pads, ointments, aromatherapy pads, pads filled with materials such as gels or beads, inflatable pads to increase or decrease pressure and pad foams comprising varying density, advanced foams and rubber;
wherein the electronic devices can use power sources comprising AC power, DC power and battery power.

12. The adjustable head and neck support device of claim 9, wherein the base, the head rest arm, the head pad, the neck pad, lock and release levers; and the neck adjustment arms are made of materials comprising plastics, PVC, ABS, wood, metal, metal, composites and combinations thereof.

13. The adjustable head and neck support device of claim 1, wherein the head pad axle is held by devices comprising hook and loop straps, buttons and loops, adhesive tape, snaps and eyelets.

14. A method of positioning an adjustable head and neck support device in an intermediate position, comprising:
(a) moving first and second neck adjustment arms by releasing first and second neck adjustment arms tabs and locking in third arm slots both on the plus and minus x-axis;
(b) inserting a tab on headrest tilt plate into a base component, wherein the tab is positioned within a second base slot;
(c) moving the neck adjustment arms with the corresponding tabs from the first slot position and locked and/or positioned in the second slot position.
(d) placing a patients neck on a cushioned neck pad and the patient's head on a cushioned head pad while the patient is in a supine position; and
(e) ending the method.

15. The method of positioning an adjustable head and neck support device of claim 14;
wherein the device utilizes equipment comprising motors, linear actuators, springs, torsion mechanisms, threaded shafts, load cells, proximity sensors, or sensor electronics or a combination thereof, to adjust the head and neck assembly;
wherein the device is digitally set comprising an electronic programmable device with software that drives the head and neck assemblies with the patients head and neck on the device controlled by a computer program based on variables comprising the patients spine curvature, x-ray measurements, a doctor's prescription, biofeedback, proximity sensors, load or force sensor readings or a combination thereof.

16. The method of positioning an adjustable head and neck support device of claim 14;
wherein the head pad axle is held by devices comprising hook and loop straps, buttons and loops, adhesive tape, snaps and eyelets.

17. The method of positioning an adjustable head and neck support device of claim 14;
an adjustable head rest assembly and an adjustable neck rest assembly are configured to attach to and move with respect to a base; and
wherein the adjustable neck rest assembly is configured to move and non-fixedly lock on a z-axis;
wherein the head rest support assembly is configured to rotate about and non-fixedly lock in a yz-plane;
wherein the adjustable neck rest assembly is configured to move and non-fixedly lock on the z-axis.

18. The method of positioning an adjustable head and neck support device of claim 14;
wherein the adjustable head rest assembly and the adjustable neck rest assembly are configured to attach to and move with respect to a base; and
wherein the adjustable neck rest assembly is configured to move and non-fixedly lock on a z-axis and to rotate about non-fixedly lock on a y-axis;
wherein the head rest support assembly is configured to rotate about non-fixedly lock on an x-axis;
wherein the adjustable head rest assembly is configured to move and non-fixedly lock on the z-axis and to rotate about the x-axis.

19. The method of positioning an adjustable head and neck support device of claim 14;
wherein the base is attached to head rest arms that rotates around base hinges; and
wherein the rotational head pad rotates about a head pad axle that rotates within the base hinges.

20. The method of positioning an adjustable head and neck support device of claim 14;
wherein the base, the head rest arms, the head pad, the neck pad, lock and release levers, the neck adjustment arms, comprise, plastics, PVC, ABS, wood, metal, metal, composites and combinations thereof.
